(19) United States
(12) Patent Application Publication
(10) Pub. No.: US 2012/0114657 A1
(43) Pub. Date: May 10, 2012

(54) Human Monoclonal Antibody Specific for Lipopolysaccharides (LPS) of Serotype IATS 01 of Pseudomonas Aeruginosa

(75) Inventors: Michael Rudolf, Zollikofen (CH); Holger Koch, Zurich (CH); Stefanie Fas, Leiden (NL)

(73) Assignee: KENTA BIOTECH AG

(21) Appl. No.: 13/262,791

(22) PCT Filed: Apr. 6, 2010
(86) PCT No.: PCT/EP2010/002158

§ 371 Date: Jan. 25, 2012

(30) Foreign Application Priority Data
Apr. 9, 2009 (EP) 09005245.7

(51) Int. Cl. A61K 39/395; G01N 33/569; C12P 21/08; C07H 21/04; C12N 15/63; A61K 31/7088; C07K 16/12; C12Q 1/68; C12N 5/16

(52) U.S. Cl. 424/142.1; 435/6.15; 435/7.32; 435/69.6; 435/326; 435/320.1; 514/44 R; 530/387.3; 530/388.15; 530/391.3; 530/391.7; 536/23.53

(57) ABSTRACT

The present invention relates to a human monoclonal antibody specific for the serotype IATS 01 of P. aeruginosa, and a hybridoma producing said monoclonal antibody. In addition, the present invention relates to pharmaceutical compositions comprising at least one antibody or at least one nucleic acid encoding said antibody.

The present invention relates to a human monoclonal antibody specific for the serotype IATS O1 of P. aeruginosa, a hybridoma producing it, nucleic acids encoding it, and host cells transfected therewith. Further, the present invention relates to methods for producing said monoclonal antibody. In addition, the present invention relates to pharmaceutical compositions comprising at least one antibody or at least one nucleic acid encoding said antibody.

P. aeruginosa is a ubiquitous gram-negative environmental bacterium found in fresh water and soil. It is a classical opportunistic pathogen that does not normally pose a threat to the immunocompetent host, who clears it by means of opsonising antibodies and phagocytosis. However, cystic fibrosis patients and immunocompromised individuals—Including burn victims, intubated patients in ICU, cancer and AIDS patients, as well as patients undergoing organ transplantation—are at particularly high risk of contracting nosocomial infections. Together with methicillin-resistant S. aureus (MRSA) and vancomycin-resistant enterococci (VRE), P. aeruginosa is responsible for up to 34% of all nosocomial infections, which have increased from 7.2/1000 patient days in 1975 to 9.8/1000 patient days in 1995. Among the most frequently observed forms of nosocomial infection are blood-stream infections and pneumonia.

An attempt was made to develop an octavalent conjugate-vaccine consisting of the 8 most relevant LPS serotypes of P. aeruginosa coupled to detoxified Toxin A of P. aeruginosa for the prevention of chronic P. aeruginosa infections in cystic fibrosis patients. Early clinical results were promising, demonstrating the induction of potent antibodies specific for the serotypes of P. aeruginosa. However, active vaccination is only possible in immunocompetent patients, as well as in predictable situations. Thus, most of the P. aeruginosa victims cannot be immunized actively with the octavalent vaccine. Due to the fact that most P. aeruginosa strains are multi-drug resistant, there is a need for an alternative therapeutic tool to treat P. aeruginosa-infected patients. One attempt is to create human monoclonal antibodies by means of classical hybridoma technology or phage display repertoire cloning.

Both methods and the antibodies created thereby show serious drawbacks.

The classical hybridoma technology (“Kohler and Milstein” approach) is based on eliciting murine B cells of desired specificity by active immunisation with an antigen of choice and immortalisation by fusion with a myeloma partner. Thereafter, the genetic information of an antibody-producing clone needs to be humanized by genetic engineering, and the antibody to be produced in a suitable expression system. Likewise, phage display repertoire cloning requires a sophisticated genetic engineering of the antibody and establishment of a suitable expression system.

It is known that murine monoclonal antibodies directed to bacterial LPS recognise epitopes other than human antibodies. Therefore, generation of monoclonal antibodies in mice followed by humanisation would not necessarily result in the isolation of antibodies with specificity relevant for the use in humans.

Furthermore, antibodies of IgM isotype are most effective due to effector mechanisms linked to IgM that are optimal for antibacterial immunity. However, to date recombinant expression of IgM antibodies has not been achieved because of the complex, pentameric form of this molecule. Consequently, expression of antibodies isolated by phage-display technology is limited to isotypes other than IgM.

Alternatively, there have been different attempts in generating human monoclonal antibodies to LPS moieties of P. aeruginosa. However, many of them lack effector functions and thus were not protective.

Accordingly, one technical problem underlying the present invention is to provide a human monoclonal antibody specific to LPS of a particular serotype of P. aeruginosa wherein the antibody exhibits high protective capacity, in particular in vivo.

The technical problem is solved by the human monoclonal antibodies as defined in the following.

According to the present invention, a human monoclonal antibody termed 216-01, specific for LPS of the P. aeruginosa serotype IATS O1 is provided wherein the variable region of the light chain of the antibody comprises at least one of SEC) ID NO:1 in the CDR1 region, SEQ ID NO: 2 in the CDR2 region and SEQ ID NO:3 in the CDR3 region, and wherein the variable region of the heavy chain of the antibody comprises at least one of SEQ ID NO:4 in the CDR1 region, SEQ ID NO:5 in the CDR2 region and SEQ ID NO:6 in the CDR3 region; or a fragment or derivative thereof capable of binding to said LPS.

According to a preferred embodiment of the present invention, a human monoclonal antibody, specific for LPS of the P. aeruginosa serotype IATS O1 is provided wherein the variable region of the light chain of the antibody comprises SEQ ID NO:1 in the CDR1 region, SEQ ID NO: 2 in the CDR2 region and SEQ ID NO:3 in the CDR3 region, and wherein the variable region of the heavy chain of the antibody comprises SEQ ID NO:4 in the CDR1 region, SEQ ID NO:5 in the CDR2 region and SEQ ID NO:6 in the CDR3 region; or a fragment or derivative thereof capable of binding to said LPS.

The present invention further provides a hybridoma capable of producing the monoclonal antibody and nucleic acids encoding the light and heavy chain of the antibody, respectively. Further, the present invention provides vectors and host cells, comprising the nucleic acid. In addition, methods for producing the monoclonal antibodies are provided. In addition, pharmaceutical compositions comprising at least one antibody and/or at least one nucleic acid and second medical uses thereof are provided.

Surprisingly, it has been found that the human monoclonal antibody according to the invention exhibit high protective capacity. In particular, the human monoclonal antibody proved to be opsonophagocytic in vitro. Even more important, the monoclonal antibody according to the present invention exhibits in vivo protective capacity as determined by the protection as well as treatment from systemic infection in the murine burn wound model.

With the human monoclonal antibodies according to the invention, opsonophagocytosis at much lower doses as well as a higher protection is achieved compared to the human monoclonal antibodies described by Collins et al. (Collins M S et al., 1990. FEMSIM 64:263-268). Furthermore, in contrast to monoclonal antibodies described in the state of the art, the human monoclonal antibody according to the invention shows both significantly better results in recognition of patient isolates and good results in opsonophagocytosis assays.

In contrast to the monoclonal antibodies described in the state of the art (Harrison F J J et al. 1997, Hybridoma 16(5):413-420; Zweerink H J et al. 1988. Infection and Immunity 56(8):1873-1879), the human monoclonal antibodies according to the invention are further generated from blood of a healthy individual actively immunized with a conjugate vaccine. It is generally known that antibodies against polysaccharides are of minor quality (i.e. low-affinity with little effector potential) because of the lack of T-cell help. Only through the use of a conjugate vaccine can valuable antibodies having high affinity with strong effector potential against polysaccharide targets be generated. Moreover, the production rate of the human monoclonal antibody according to the invention is higher compared to the production rate of monoclonal antibodies described in the state of the art (Zweerink H J et al. 1988. Infection and Immunity 56(8):1873-1879).

According to the present invention, the antibody is specific for the LPS of P. aeruginosa serotype IATS O1 and exhibits opsonophagocytic activity at concentrations as low as 0.1 ng/ml, preferably at a concentration as low as 0.5 ng/ml as determined using fluorescence-conjugate bacteria. No prior art antibody has been reported exhibiting an opsonophagocytic activity at this low dosage.

The antibody of the invention is specific for the LPS of P. aeruginosa serotype IATS O1 and exhibits a half maximum opsonophagocytic activity at concentrations between 1.7 and 4.3 ng/ml (95% confidence interval), specifically at a concentration of about 2.7 ng/ml.

The invention also contemplates an antibody that specifically binds to the LPS of Pseudomonas aeruginosa serotype IATS 01 with an avidity of:

1.03 10⁸ M⁻¹+/−3.41×10⁷ M⁻¹.

The monoclonal antibody according to the present invention recognizes clinical isolates with high specificity. 10 of 10 samples of patients infected with P. aeruginosa of the IATS O1 serotype were identified using this antibody. Without being bound by theory, it is assumed that the monoclonal antibody is capable of recognizing all P. aeruginosa strains of IATS O1 known in the prior art. This property renders the antibody particularly useful for diagnosis and therapy. Thus, the antibody according to the present invention exhibits an insurmountable reliability.

The term “human monoclonal antibody” as used herein encompasses any partially or fully human monoclonal antibody independent of the source from which the monoclonal antibody is obtained. The production of the human monoclonal antibody by a hybridoma is preferred. The monoclonal antibody may also be obtained by genetic engineering and in particular CDR grafting of the CDR segments as defined in the claims onto available monoclonal antibodies by replacing the CDR regions of the background antibody with the specific CDR segments as defined in the claims.

“CDR region” is the term used for the complementarity determining region of an antibody, i.e. the region determining the specificity of an antibody for a particular antigen. Three CDR regions (CDR1 to CDR3) on both the light and heavy chain are responsible for antigen binding.

The CDRs were determined by applying the Kabat numbering as shown at http://www.bioinf.org.uk/abs/seqtest.html.

The positions of the CDR regions within the heavy chain are as follows:

CDR1 region amino acids 31 to 35 within the V_{H} exon,
CDR2 region amino acids 50 to 65 within the V_{H} exon,
CDR3 region amino acids 95 and following amino acids within the V_{H} exon.

The positions of the CDR regions are independent from the class of antibody, i.e. IgM, IgA or IgG.

The positions of the CDR regions of the kappa light chain are as follows:

CDR1 region amino acids 24 to 34 within the V_{χ} exon,
CDR2 region amino acids 50 to 56 within the V_{χ} exon,
CDR3 region amino acids 89 and following amino acids within the V_{χ} exon.

The positions of the CDR region within the lambda type light chain are as follows:

CDR1 region amino acids 24 to 34 within the Vλ exon,
CDR2 region amino acids 50 to 56 within the Vλ exon,
CDR3 region amino acids 89 and following amino acids within the Vλ exon.

Amino acid alignments of the V_{H}, V_{χ} and Vλ exon can be obtained from V base index. (http://vbase.mrc-cpe.cam.ac.uk/).

The term “serotype” means any known serotype of P. aeruginosa. A concordance table of the different nomenclatures presently used for different P. aeruginosa serotypes is shown in table I in the specification.

The term “fragment” means any fragment of the antibody capable of binding to the LPS serotype. The fragment has a length of at least 10, preferably 20, more preferably 50 amino acids. Examples of suitable antibody fragments include divalent fragments, e.g., F(ab)₂, F(ab′)₂, monovalent fragments, e.g., Fab, Fab′, Fv, single chain recombinant forms of the foregoing, and the like. Antibody fragments may be glycosylated, for example containing carbohydrate moieties in the antibody variable regions. It is preferred that the fragment comprises the binding region of the antibody. It is preferred that the fragment is a Fab or F(ab′)₂ fragment or a mixture thereof.

The term “derivative” encompasses any muteins of the human monoclonal antibody differing by the addition, deletion, and/or substitution of at least one amino acid. Preferably, the derivative is a mutein of the human monoclonal antibody wherein the mutein carries at least one conservative substitution in any of the CDR's in the heavy chain and/or light chain as indicated in the claims. More preferably, the mutein has not more than 5, not more than 4, preferably not more than three, particularly preferred not more than 2 conservative substitutions. The capacity of the fragment or derivative of the antibody to bind to the particular LPS serotype is determined by direct ELISA as described in the material and methods section: the particular LPS is immobilized on the solid phase of ELISA plates. Antibody fragments or derivative of the antibodies are incubated with the immobilized LPS, and bound antibodies or derivatives thereof are visualized by a suitable enzyme-conjugated secondary antibody.

In accordance with the present invention, the term “conservative substitution” means a replacement of one amino acid belonging to a particular physico-chemical group with an amino acid belonging to the same physico-chemical group. The physico-chemical groups are defined as follows:

The group of non-polar amino acids comprises: glycine, alanine, valine, leucine, isoleucine, methionine, proline, phenylalanine, and tryptophan. The group of amino acids having uncharged polar side chains comprises asparagine, glutamine, tyrosine, cysteine, and cystine. The physico-chemical group of amino acids having a positively charged polar side chain comprises lysine, arginine, and histidine. The physico-chemical group of amino acids having a negatively charged polar side chain comprises aspartic acid and glutamic acid, also referred to as aspartate and glutamate.

According to the present invention, an antibody specific for LPS of the P. aeruginosa serotype IATS O1 is provided as outlined above.

According to a further embodiment the present invention provides a human monoclonal antibody specific for LPS or the P. aeruginosa LPS serotype IATS O1 wherein the variable region of the light chain of the antibody has the amino acid sequence of SEQ ID NO:7 and the variable region of the heavy chain has the amino acid sequence of SEQ ID N0:8; or a variant of said antibody capable of binding said LPS wherein the variable region of the amino acid sequence of the light chain of the antibody is at least 85% homologous, preferably at least 90% homologous, more preferably at least 95% homologous to SEQ ID NO:7 and the amino acid sequence of the variable region of the heavy chain of the antibody is at least 85% homologous, preferably at least 90% homologous, more preferably 95% homologous to SEQ ID NO:8.

The term “homology” known to the person skilled in the art designates the degree of relatedness between two or more polypeptide molecules, which is determined by the agreement between the sequences. The percentage “homology” is found from the percentage of homologous regions in two or more sequences, taking account of gaps or other sequence features.

The homology of mutually related polypeptides can be determined by means of known procedures. As a rule, special computer programs with algorithms taking account of the special requirements are used. Preferred procedures for the determination of homology firstly generate the greatest agreement between the sequences studied. Computer programs for the determination of the homology between two sequences include, but are not limited to, the GCG program package, including GAP (Devereux J et al., Nucleic Acids Research 12 (12): 387 (1984); Genetics Computer Group University of Wisconsin, Madison (WI); BLASTP, BLASTN and FASTA (Altschul S et al., J. Molec. Biol. 215: 403-410 (1990)). The BLAST X program can be obtained from the National Centre for Biotechnology Information (NCBI) and from other sources (BLAST Handbook, Altschul S at al., NCB NLM NIH Bethesda Md. 20894; Altschul S et al. J. Mol. 215: 403-410 (1990)). The well-known Smith-Waterman algorithm can also be used for the determination of homology.

Preferred parameters for the sequence comparison include the following:
Algorithm: Needleman and Wunsch, J. Mol. Biol. 48 (1970), 443-453
Comparison matrix: BLOSUM62 from Henikoff & Henikoff, PNAS USA 89 (1992), 10915.10919
Gap penalty: 12
Gap-length penalty: 2

The GAP program is also suitable for use with the above parameters. The above parameters are the standard parameters (default parameters) for amino acid sequence comparisons, in which gaps at the ends do not decrease the homology value. With very small sequences compared to the reference sequence, it can further be necessary to increase the expectancy value to up to 100,000 and in some cases to reduce the word length (word size) down to 2.

Further model algorithms, gap opening penalties, gap extension penalties and comparison matrices including those named in the Program Handbook, Wisconsin Package, Version 9, September 1997, can be used. The choice will depend on the comparison to be performed and further on whether the comparison is performed between sequence pairs, where GAP or Best Fit are preferred, or between one sequence and a large sequence database, where FASTA or BLAST are preferred.

An agreement of 85% determined with the aforesaid algorithms is described as 85% homology. The same applies for higher degrees of homology.

In preferred embodiments, the muteins according to the invention have a homology of 85% or more, e.g. more than 90% or 95%.

It is further preferred that the light chain of the human monoclonal antibody according to the present invention is of the kappa or lambda type. Particularly preferred, the light chain is of the kappa type. The light chain may be either a naturally occurring chain including a naturally rearranged, a genetically modified or synthetic type of light chain. If the antibody according to the present invention being specific to IATS O1 is of the kappa type, then it is preferred that the light chain be derived from germ line DPK18 (http://vbase.mrc-cpe.cam.ac.uk/).

According to a further preferred embodiment, the heavy chain of the human monoclonal antibody of the present invention is selected from all human isotypes, namely IgM, IgA, or IgG. Preferably, the heavy chain is of the IgM type. If the antibody is of the IgM type, then it exhibits the advantageous properties of high avidity for P. aeruginosa LPS, effectively binds the complement and thus mediates either direct killing of bacteria, and/or efficiently opsonizes bacteria for phagocytosis. Further, IgM is resistant to the proteolytic degradation by P. aeruginosa elastase, whereas other isotypes like IgG or IgA can be degraded. IgM antibodies are effective in low amounts. 1 to 4 μg per mouse were protective in the murine burn wound sepsis model.

It is preferred that the variable heavy chain be derived from germ line VH3-11 (http://vbase.mrc-cpe.cam.ac.uk/). The light chain and heavy chain may either be covalently linked as a single-chain antibody (e.g. bivalent scFv, bifunctional scFv and bispecific scFv) or non-covalently linked with each other.

According to a preferred embodiment of the present invention, the human monoclonal antibody consists entirely of human amino acid sequence.

“Consists entirely of human amino acid sequence” means that the amino acid sequence of the human monoclonal antibody is derived from a human germ line. This may be obtained in different ways. For example, the human monoclonal antibody consisting of human amino acid sequence can be obtained from a hybridoma wherein the B-cell is a human B-cell. Alternatively, the human monoclonal antibody may be obtained by CDR grafting of the CDR regions as indicated in the claims onto available human monoclonal antibodies thereby producing a human monoclonal antibody specific for a P. aeruginosa LPS serotype in accordance with the present invention.

The entirely human amino acid sequence of the human monoclonal antibody prevents the occurrence of undesired adverse effects such as rejection reactions or anaphylactic shock.

Further preferred, the human monoclonal antibody exhibits human antigen recognition. “Human antigen recognition” means that the antigen recognition by the human monoclonal antibody according to the present invention is essentially mediated through human derived antigen-specific variable regions of the antibody, thus identical to the recognition of antigen by a healthy human individual. In particular it is also required that the Fc portions of the heavy and light chain of the human monoclonal antibody are of human type in order to ensure interaction with human complement system, and to reduce the risk of generation of so called HAMA (human anti-mouse-antibodies).

According to a further preferred embodiment, the human monoclonal antibody of the present invention is obtainable from a human B-cell or a hybridoma obtained by fusion of said human B-cell with a myeloma or heteromyeloma cell.

Human B-cells may be obtained by immunization of healthy individuals or patients and subsequent removal of blood samples from which human B-cells can be isolated in a known manner (Current Protocols in Immunology. Chapter 7.1. Isolation of whole mononuclear cells from peripheral blood and cord blood. Published by Wiley & sons, Eds: J C Coligan et al.). The human B-cell may be fused to a myeloma or heteromyeloma to produce a hybridoma in accordance with known techniques according to the classical Kohler and Milstein approach. Suitable myeloma cells are derivatives of P3X63 such as P3X63Ag8.653 (ATCC CRL-1580) or SP2/0 (ATCC CRL-1646). Suitable heteromyeloma cells are e.g. F3B6 (ATCC HB-8785). The resulting hybridoma may be selected according to known procedures. The hybridomas are cultured in a suitable culture medium and the produced antibody is recovered from the supernatant.

Further, the present invention provides nucleic acids encoding the heavy chain and light chain, respectively, of the human monoclonal antibody of the present invention. The nucleic acid may be a naturally occurring nucleic acid either derived from the germ line or from rearrangement occurring in B-cells, alternatively the nucleic acids may be synthetic. Synthetic nucleic acids also include nucleic acids having modified internucleoside bonds including phosphothioester to increase resistance of the nucleic acids from degradation. The nucleic acid may be genetically engineered or completely synthetically produced by nucleotide synthesis.

The present invention further provides vectors comprising at least one nucleic acid encoding the light chain of the human monoclonal antibody of the present invention and/or at least one nucleic acid encoding the heavy chain of the human monoclonal antibody of the present invention. The nucleic acids may be either present in the same vector or may be present in the form of binary vectors. The vector preferably comprises the promoter operatively linked to the nucleic acid in order to facilitate expression of the nucleic acid encoding the light and/or heavy chain. Preferably, the vector also includes an origin for replication and maintenance in a host cell. The vector may also comprise a nucleotide sequence encoding a signal sequence located 5′ of the nucleic acid encoding the light chain or heavy chain. The signal sequence may facilitate secretion of the encoded chain into the medium.

Preferably, the vector is derived from adenoviruses, vaccinia viruses, baculoviruses, SV 40 viruses, retroviruses, plant viruses or bacteriophages such as lambda derivatives or M13. The particularly preferred vector is a vector containing the constant regions of human Ig heavy chains and human light chains, such as the integrated vector system for eukaryotic expression of immunoglobulins described by Persic et al. (Persic et al. 1997. Gene. 187(1) 9-18).

The vector may further comprise a His-tag coding nucleotide sequence resulting in the expression of a construct for producing a fusion product with a His-tag at the N-terminus of the light and/or heavy chain of the human monoclonal antibody, which facilitates purification of the protein at a nickel column by chelate formation.

Further, the present invention provides host cells comprising the vector and/or the nucleic acid suitable for the expression of the vector. In the art, numerous prokaryotic and eukaryotic expression systems are known wherein eukaryotic host cells such as yeast cells, insect cells, plant cells and mammalian cells, such as HEK293-cells, PerC6-cells, CHO-cells, COS-cells or HE LA-cells and derivatives thereof are preferred. Particularly preferred are human production cell lines. It is preferred that the transfected host cells secrete the produced antibody into the culture medium. If intracellular expression is achieved, then renaturation is performed in accordance with standard procedures such as e.g. Benetti P H et al., Protein Expr Purif August; 13:283-290, (1998).

The present invention also provides methods for producing the human monoclonal antibody. In one embodiment the human monoclonal antibody is produced by culturing the above-described hybridoma. The produced monoclonal antibody is secreted into the supernatant and can be purified from it by applying conventional chromatographic techniques.

Alternatively, the human monoclonal antibody is produced by the host cell comprising a vector according to the present invention and culturing the host cell under conditions suitable for recombinant expression of the encoded antibody chain. Preferably, the host cell comprises at least one nucleic acid encoding the light chain and at least one nucleic acid encoding the heavy chain and is capable of assembling the human monoclonal antibody such that a 3-dimensional structure is generated which is equivalent to the 3-dimensional structure of a human monoclonal antibody produced by a human B-cell. If the light chain is produced separately from the heavy chain, then both chains may be purified and subsequently be assembled to produce a human monoclonal antibody having essentially the 3-dimensional structure of a human monoclonal antibody as produced by a human B-cell.

The human monoclonal antibody may also be obtained by recombinant expression of the encoded light and/or heavy chain wherein the nucleic acid is produced by isolating a nucleic acid encoding a human monoclonal antibody in a known manner and grafting of the nucleic acid sequence encoding the CDR's as defined in the claims onto the isolated nucleic acid.

According to a further preferred embodiment, the human monoclonal antibody according to the present invention is modified. The modifications include the di-, oligo-, or polymerization of the monomeric form e.g. by cross-linking using dicyclohexylcarbodiimide. The thus produced di-, oligo-, or polymers can be separated from each other by gel filtration. Further modifications include side chain modifications, e.g. modifications of ε-amino-lysine residues, or amino and carboxy-terminal modifications, respectively. Further modifications include post-translational modifications, e.g. glycosylation and/or partial or complete deglycosylation of the protein, and disulfide bond formation. The antibody may also be conjugated to a label, such as an enzymatic, fluorescent or radioactive label.

The present invention further provides pharmaceutical compositions comprising at least one human monoclonal antibody and/or at least one nucleic acid encoding a light and/or heavy chain of the human monoclonal antibody.

The pharmaceutical composition may further comprise pharmaceutically acceptable ingredients known in the art.

Preferably, the pharmaceutical compositions are applied for the treatment of diseases caused by P. aeruginosa in infections such as blood-stream infection, pneumonia, chronic bronchitis, local infections including wound infections and invasive infections of joints, mainly in immunocompromised patients and/or in patients with compromised respiratory function. The pharmaceutical compositions are further intended for but not limited to the prophylaxis and/or treatment of hospital-acquired (nosocomial) infections. Since the main victims of P. aeruginosa infections are cystic fibrosis patients, burn victims, intubated patients, patients in surgical and/or medical intensive care units, cancer and AIDS patients, immunocompromised patients, immunosuppressed patients, diabetic patients, as well as intravenous drug abusers, the pharmaceutical compositions are in particular intended for prophylaxis and/or treatment of diseases caused by P. aeruginosa in said group of patients.

The pharmaceutical composition may further comprise antibiotic drugs, preferably coupled to the new monoclonal antibody.

The pharmaceutical compositions comprise the new monoclonal antibody in a concentration range of 0.1-30 mg/kg body weight.

The pharmaceutical compositions may be administered in any known manner such as intravenous, intra-muscular, intra-dermal, subcutaneous, intra-peritoneal, topical, intranasal administration, or as inhalation spray.

The present invention also provides a test kit for the diagnosis of P. aeruginosa infections comprising at least one human monoclonal antibody of the present invention and optionally further suitable ingredients for carrying out a diagnostic test. Suitable ingredients for carrying out such diagnostic test are well known in the art. Particularly useful examples for suitable ingredients are buffer solutions, such as a buffer solution with an osmolality within a range of 280-320 mOsm/l and a pH value within a range of pH 6-8, a buffer solution containing chelating agents, a buffer solution containing monovalent or bivalent cations with the total cation concentration of the buffer composition ranging from about 0.02 M to about 2.0 M, or a buffer solution containing animal or human derived serum at a concentration between 0.01% and 20%.

The test kit is suitable for the specific reliable diagnosis of a P. aeruginosa infection. A test assay may be based on a conventional ELISA test in liquid or membrane-bound form. The detection may be direct or indirect as known in the art wherein the antibody is optionally conjugated to an enzymatic, fluorescent or radioactive label.

The following examples illustrate the invention but are not intended to limit the scope of the present invention. Further embodiments will be apparent for the person skilled in the art when studying the specification and having regard to common general knowledge.

### BRIEF DESCRIPTION OF THE FIGURES

FIG. 1 relates to DNA and amino acid sequence of 216-O1 heavy chain variable region. The CDR1 region of 216-01 is at positions 31 to 35, the CDR2 region of 216-01, is at positions 50 to 66, and the CDR3 region of 216-01 is at positions 99 to 104.

FIG. 2 relates to DNA and amino acid sequence of 216-O1 kappa light chain variable region. The CDR1 region of 216-01 is at positions 24 to 39, the CDR2 region of 216-01, is at positions 55 to 61, and the CDR3 region of 216-01 is at positions 94 to 101.

FIG. 3 relates to the recognition pattern of LPS isolated from P. aeruginosa strains by the monoclonal antibody 216-O1. The binding of 216-O1 was determined by ELISA.

FIG. 4a relates to the recognition of P. aeruginosa reference strains (serotype O1-O17) by the monoclonal antibody 216-O1. FIG. 4b relates to the recognition pattern of clinical P. aeruginosa isolates by the monoclonal antibody 216-O1 and two other known antibodies (MAb C1 and MAb C2). The binding of the antibodies was determined by whole cell ELISA (for source of antibodies, see page 19, example: whole cell ELISA)

FIG. 5 relates to the opsonophagocytotic activity of the monoclonal antibody 216-O1 and two other known antibodies (MAb C1 and MAb C2) directed against P. aeruginosa serotype IATS O1.

FIG. 6 relates to the pharmocodynamics of the monoclonal antibody 216-O1 in mice. The in vivo protective capacity of 216-O1 was assessed in a murine burn wound sepsis model. Different doses of 216-O1 were administered i.v. to NMRI mice. Survival rates are shown up to 96 h after challenge (FIG. 6A) and a summary of 3 experiments three days after challenge is shown (FIG. 6B).

### MATERIAL AND METHODS

The Following Material and Methods have been Used in the Examples:

### Determination of LPS-Specificity and Quantification of IgM

For screening and analysis of antibodies in cell culture supernatants, an ELISA was performed as described elsewhere (Cryz, S. J. et al., 1987. J. Clin. Invest. 80(1):51-56) with some alterations. Briefly, P. aeruginosa lipopolysaccharide (LPS) (produced in house) stock solutions were prepared at a concentration of 2 mg/ml in 36 mM triethylamine or in H₂O. For coating, the solution was diluted to 10 μg/ml in PBS. This solution was mixed with an equal volume of 10 mg/ml methylated human serum albumin (HSA; produced in house as follows: 2 g of lyophilized HSA was dissolved in 200 ml absolute methanol. After adding 1.68 ml of 37% HCl, the solution is stored for at least 3 day at room temperature in the dark with occasional shaking. The precipitate is collected by 10 min centrifugation (4500 rpm, GS1 rotor), and washed twice with absolute methanol and twice with anhydrous ether by suspending the pellet in the solvent. The precipitate is dried for 2 hours in a desiccator and the dry pellet is suspended in H₂O, and stored in aliquots at −20° C. NUNC® ELISA plates were coated with 100 μl/well LPS-HSA solution overnight at room temperature. After washing the plates 3× with 300 μl PBS pH 7.4 (produced in house) containing 0.05% Tween20 (#93773; Fluka Chemie AG, Switzerland) (PBS-T), cell culture supernatants were diluted 1:2 in PBS and incubated for 2 hours at room temperature. After washing the plates 3× with PBS-T, bound antibodies were detected with horseradish peroxidase-conjugated goat anti-human IgM antibody (#074-1003; KPL; Kirkegaard & Perry Laboratories, Inc. Gaithersburg, Md.) diluted 1:2000-1:4000 in PBS-T. The plates were incubated for 1 hour at room temperature, and washed 3× with PBS-T. Antibody-binding was visualized by adding 100 μl/well OPD substrate solution (0.4 mg/ml Orthophenyldiamine in 0.1M sodium-citrate buffer containing 0.012% (v/v) H₂O₂). Color reaction was stopped after 2-3 min by the addition of 50 μl/well 1 M HCl. Optical density was read on an ELISA reader at 490 nm using Softmax Pro® software.

For quantification of IgM in cell culture supernatants, ELISA plates were coated with 1 μg/ml unconjugated goat anti-human IgM antibody in PBS overnight at 4° C. Plates were washed 3× with PBS-T, and cell supernatants and standards were incubated in 2-fold dilutions. As a standard, a purified human antibody was used starting at a concentration of 0.5 μg/ml. All dilutions were done in PBS-T. Plates were incubated for 2 hours at room temperature. After washing the plates 3× with PBS-T, bound antibodies were detected with horseradish peroxidase-conjugated goat anti-human IgM antibody (KPL) diluted 1:2000-1:4000 in PBS-T. The plates were incubated for 1 hour at room temperature, and washed 3× with PBS-T. Antibody-binding was visualized by adding 100 μl/well OPD substrate solution. Color reaction was stopped after about 1 min by the addition of 50 μl/well 1 M HCl. Optical density was read on an ELISA reader at 490 nm using Softmax Pro® software.

### Determination of Avidity

The avidity was determined using an inhibition assay in which is investigated how the addition of free LPS to the antibody influences its binding to the coated LPS. The avidity is the reciprocal value of the concentration of free LPS (in mol/L) which confers 60% inhibition of the signal of the antibody to only coated LPS. This was calculated using the Reed-Münch method (Reed L. J. and Muench H., Am J of Hygiene (27), 493-497 (1938))

Plates were coated with LPS as described above (Determination of LPS specificity). After washing the plates 3× with 300 μl PBS pH 7.4 (produced in house) containing 0.05% Tween20 (#93773; Fluka Chemie AG, Switzerland) (PBS-T), the antibody was added. As a reference, a dilution row of antibody in PBS was used. In addition different concentrations of free LPS (in H₂O) were added in a second dilution row using a constant concentration of 216-01. The plates were incubated 2 hours at room temperature and subsequently washed 3× with PBS-T. Plate-bound antibodies were detected with horseradish peroxidase-conjugated goat anti-human IgM antibody (#074-1003; KPL; Kirkegaard & Perry Laboratories, Inc. Gaithersburg, Md. or #62-7500 Zymed, Invitrogen, Carlsbad) diluted 1:2000 or 1:4000 respectively in PBS-T. The plates were incubated for 1 hour at room temperature, and washed 3× with PBS-T. Antibody-binding was visualized by adding 100 μl/well OPD substrate solution (0.4 mg/ml Orthophenyldiamine in 0.1M sodium-citrate buffer containing 0.012% (v/v) H₂O₂). Color reaction was stopped after 2-3 min by the addition of 50 μl/well 1 M HCl. Optical density was read on an ELISA reader at 490 nm using Softmax Pro® software.

### Sequence Analysis

RNA of hybridoma cells was isolated by using RNeasy-Kit from Qiagen. cDNA was synthesized using reverse transcriptase (Superscript II, Invitrogen and Primescript, Takara Bio Inc.). Using a human IgG and IgM library primer set (#F2000, Progen), designed for the amplification of human rearranged IgG and IgM variable domain coding regions, the subgroup of the heavy and light chain was determined. Specific forward primers in the leader sequences were designed and used in combination with constant primers for amplifying the variable regions by PCR and sequencing. For sequencing, in addition forward primers in the variable regions were designed to confirm the sequence. Sequencing was performed at Microsynth AG (Balgach, Switzerland).

For PCR and sequencing the following primers were used (table III): reverse constant IgM (IgM con): 5′-AAG GGT TGG GGC GGA TGC ACT-3′; reverse constant Kappa (Kappa rev): 5′-GAA GAC AGA TGG TGC AGC CAC AG-3′. As forward primer for the heavy chain VH3: 5′-ATG GAG TTT GGG CTG AGC TG-3′ and for the light chain Leader 1: 5″-CAA TGA GGC TCC CTG CTC AG-3′ were used.

For sequencing, in addition, the following forward primers have been designed and used for the heavy chain HC CDR2-3: 5″-AGT CTG AGA GCC GAG GAC AC-3′ and for the light chain LC CDR2-3: 5′-ACA GAT TCA GCG GCA GTG G-3′.

The CDRs were determined by applying the Kabat numbering via http://www.bioinf.org.uk/abs/seqtest.html.

Sequences were compared with existing germline sequences using the V-Base DNAPLOT software (http://vbase.mrc-cpe.cam.ac.uk/).

TABLE I
IATS Serotypes of P. aeruginosa vaccination strains

| IATS Serotype | Specification |
|---|---|
| O1 | PA53 (IT4) |
| O3 | 6510 (Habs3) |
| O4 | 6511 (Habs4) |
| O5 | Fisher 7 (IT7) |
| O6 | PA220 (IT1) |
| O10 | Fisher 5 (IT5) |
| O11 | Fisher 2 (IT2) |
| O16 | E576 (IT3) |

TABLE II
Clinical isolates of P. aeruginosa serotype IATS O1

| Isolate | Origin |
|---|---|
| PEG12 | Clinical Isolate |
| | Basel |
| PEG37 | Clinical Isolate |
| | Basel |
| 487/T421 | In house strain |
| | collection |
| 615/T341 | In house strain |
| | collection |
| PEG3 | Clinical Isolate |
| | Basel |
| PEG7 | Clinical Isolate |
| | Basel |
| PEG9 | Clinical Isolate |
| | Basel |
| 2309.07 | Clinical Isolate |
| | Bern |
| 2309.24 | Clinical Isolate |
| | Bern |
| 2310.20 | Clinical Isolate |
| | Bern |
| PEG4 (IATS O1) | Clinical Isolate |
| | Basel |

These P. aeruginosa isolates were obtained from patients from various sources such as urine or the respiratory tract.

### Whole Cell ELISA

P. aeruginosa reference strains O1-O17 and bacteria from different clinical isolates (see Table II) were used in this assay. One P. aeruginosa strain of each serotype O1-O17 was tested as reference strain (ATCC—American Type Culture Collection): Reference strain O1 (ATCC 33348), reference strain O2 (ATCC 33356), reference strain O3 (ATCC 33350), reference strain O4 (ATCC 33351), reference strain O5 (ATCC 33352), reference strain O6 (ATCC 33354), reference strain O7 (ATCC 33353), reference strain O8 (ATCC 33355), reference strain O9 (ATCC 33356), reference strain O10 (ATCC 33357), reference strain O11 (ATCC 33358), reference strain O12 (ATCC 33359), reference strain O13 (ATCC 33360), reference strain O14 (ATCC 33361), reference strain O15 (ATCC 33362), reference strain O16 (ATCC 33363) and reference strain O17 (ATCC 33364).

Bacteria were grown in Brain Heart Infusion (BHI) medium at 37° C. to an optical density of 1 at 550 nm, and fixed with 37% Formalin (final concentration of formalin: 0.5%) overnight at 37° C. The fixed bacteria were diluted 1:50 in PBS and 100 μl immobilized on ELISA plates overnight at room temperature. After blocking the plates with 120 μl PBS containing 0.5% bovine serum albumin (BSA), for 30 min at 37° C., 100 μl of the hybridoma supernatant containing the monoclonal antibody 216-O1 was incubated with the fixed bacteria for 90 min at 37° C. Alternatively, the isolates were incubated with medium alone or a control antibody (data not shown). After washing the plates 3× with PBS-T (PBS, 0.5% Tween-20), bound antibodies were detected with horseradish peroxidase-conjugated goat anti-human IgM antibody (#074-1003; KPL; Kirkegaard & Perry Laboratories, Inc. Gaithersburg, Md.) diluted 1:2000-1:4000 In PBS-T. The plates were incubated for 1 hour at 37° C., and washed 3× with PBS-T. Antibody-binding was visualized by adding 100 μl/well OPD substrate solution (0.4 mg/ml Orthophenyldiamin in 0.1M sodium-citrate buffer containing 0.012% (V/V) H₂O₂). Color reaction was stopped after 2-3 min by the addition of 50 μl/well 1 M NCI. Optical density was read on an ELISA reader at 490 nm using Softmax Pro® software.

For the comparison experiments, additional anti-P. aeruginosa LPS serotype IATS O1 secreting cell lines 9D10 and C5D5 as described in U.S. Pat. No. 4,834,975 (Siadak) were ordered from ATCC, antibody produced (MAb C1 (9D10) and MAb C2 (C5D5), respectively) and compared with 216-O1.

### Opsonophagocytosis Assay

In order to determine the biological activity, the monoclonal antibody 216-O1 was tested for its opsonophagocytic activity. For this purpose, P. aeruginosa bacteria of the serotype IATS O1 (strain PA53) were grown in TSBG (30 g/l Tryptic Soy Broth containing 1% (w/v) Glucose) medium overnight. After washing twice the bacteria with 20 ml 0.1M Bi-Carbonate buffer, pH 8.0, the bacterial pellet was resuspended in 5 ml 0.1 M Bi-Carbonate buffer, pH 8.0. 50 μl of 5- (and -6)-carboxyfluorescein, succinimidyl ester (5(6)-FAM SE); Molecular Probes, Eugene, Oreg.; 10 mg/ml in dimethyisulfoxide) were added, and incubated at 37° C. for 1 hour. Bacteria were fixed by the addition of 100 μl 37% formaldehyde and incubation overnight at 37° C. To remove the unconjugated dye, bacteria were washed 6 times with 20 ml cold sterile PBS, resupended in 5 ml and diluted to OD₅₅₀ₙₘ=1 in PBS. The labeled bacteria were stored in aliquots at −80° C. until use. For the assay, an aliquot of the bacteria was diluted 1:50 in HBSS-BSA (Hanks balanced salt solution containing 0.1% BSA). 70 μl of the bacteria were mixed with 30 μl of different dilutions of hybridoma cell culture supernatant containing the monoclonal antibody 216-O1, or a non-specific monoclonal control antibody respectively (data not shown). In addition, 20 μl of baby rabbit serum (Charles River Laboratories, Germany) was added as a source of complement or heat inactivated complement (1 h 56° C.) as control. After 30 min of incubation at 37° C., 60 μl of differentiated HL-60 cells (the promyelocytic cell line HL-60 was differentiated into granulocytic cells by incubating the cells for 4 days in Iscoves Modified Dulbecco's Medium (IMDM; Sigma) supplemented with 20% (v/v) Fetal Calf Serum and 100 mM di-methyl-formamide) were added to the opsonized bacteria to obtain a final concentration of 1.3×10⁶ cells/ml. After incubating for 90 min at 37° C. on a shaker, 2 ml of cell wash buffer (PBS-containing 0.02% (v/v) azide; Becton Dickenson) and 100 μl of trypane blue solution (#T8154, Sigma) were added for 1 min for quenching. After centrifugation for 5 min at 350×g, the cell pellet was resuspended in about 200 μl cell wash buffer and analyzed by flow cytometry. Positive opsonphagocytotic activity was determined by analyzing the green fluorescence of the HL-60 cells in comparison with background staining. Background staining was determined by incubating fluorescein-conjugated bacteria in the presence of complement with HL-60 cells.

In Vivo Protection of P. aeruginosa Infected Mice

### Murine Burn Wound Model

The in vivo protective capacity of 216-01 was determined in the murine burn wound sepsis model. NMRI-Mice (18-20 g; Charles River Laboratories) received 0.1 to 1.5 mg/kg monoclonal antibody 216-01 in a volume of about 0.1 ml intravenously 2 hours prior to challenge. As control, 1.5 mg/kg of unspecific control (ctr) antibody was injected. For challenge, groups of 10 female mice were anesthetized with Ketamine (Narketan; Vetoquinola G)/Xalzine (Xylasol; Dr. E. Graeub A F) with 66 mg/kg Ketamine and 13.2 mg/kg Xylazine. Immediately before the burn, mice were also kept in 5% isolfurane for 2-3 min. The mice were subjected to a 10 second ethanol burn over a 2 cm² area of the back. 2.5-5×10⁷ cfu/mouse of the challenge organisms (P. aeruginosa IATS O1; PA53, see table 1) suspended in 0.5 ml PBS were injected immediately subcutaneously into the burned area. The animals were treated with 0.3 mg/kg Temgesic (analgesic) s.c. 2× a day and survival was monitored 3× daily up to 96 h after the challenge.

### EXAMPLES

### Example 1

### DNA and Amino Acid Sequences of 216-O1

The antibody specificity is determined by the DNA- and amino acid-sequence, respectively. DNA sequences of the variable fragments of the heavy and light chains were determined. Briefly, total RNA of the hybridoma cells was isolated, and reverse transcribed into complete cDNA. Using Cκ and Cμ-specific primers in combination with forward primers in the leader sequence, the IgM and Kappa variable regions and part of the constant regions were amplified by PCR. The PCR fragments were then cleaned up by excision from agarose gels, and used as templates for sequencing with the primers depicted in Table III.

### Table III

### Primers Used for PCR-Amplification and Sequencing of the Variable Regions of IgM Heavy Chain and Kappa Light Chain of 216-O1

| Primer | HC/LC | Sequence, SEQ ID Nos 11 to 16 | Application |
|---|---|---|---|
| IgM con | HC | 5′-AAG GGT TGG GGC GGA TGC ACT | PCR, Sequencing |
| VH3 | HC | 5′-ATG GAG TTT GGG CTG AGC TG | PCR, Sequencing |
| HC CDR2-3 | HC | 5′ AGT CTG AGA GCC GAG GAC AC | Sequencing |
| Kappa rev | LC | 5′ GAA GAC AGA TGG TGC AGC CAC AG | PCR, Sequencing |
| Leader 1 | LC | 5′ CAA TGA GGC TCC CTG CTC AG | PCR, Sequencing |
| LC CDR2-3 | LC | 5′ACA GAT TCA GCG GCA GTG G | Sequencing |

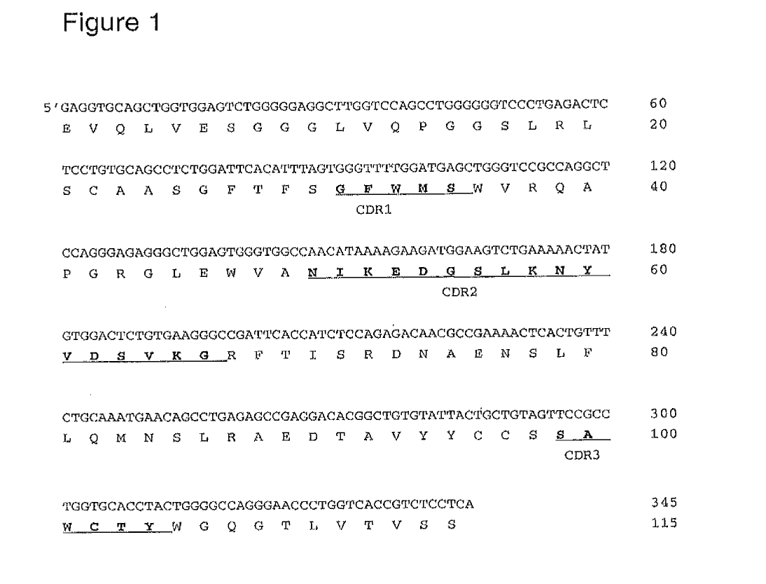

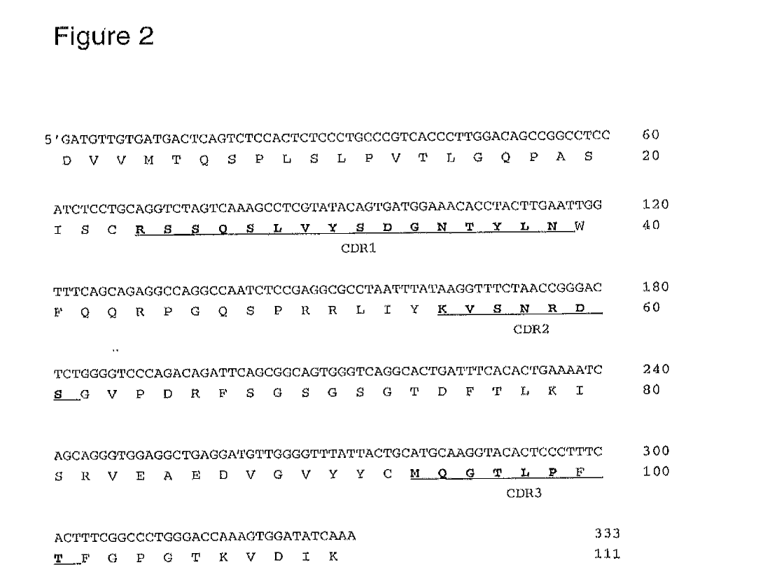

The sequences of the variable regions were subsequently compared with the Vbase Index (http://vbase.mrc-cpe.cam.ac.uk/). The comparison with germline sequences showed that the light chain has highest similarity with the DPK18 and the heavy chain with VH3-11 germline sequences. The DNA sequences and amino acid sequences of the variable region IgM heavy chain and Kappa light chain of 216-O1 are depicted in FIGS. 1 and 2.

### Example 2

### Recognition of Isolated LPS from P. aeruginosa and of Clinical Isolates of P. aeruginosa Serotype IATS O1 by Monoclonal Antibody 216-O1

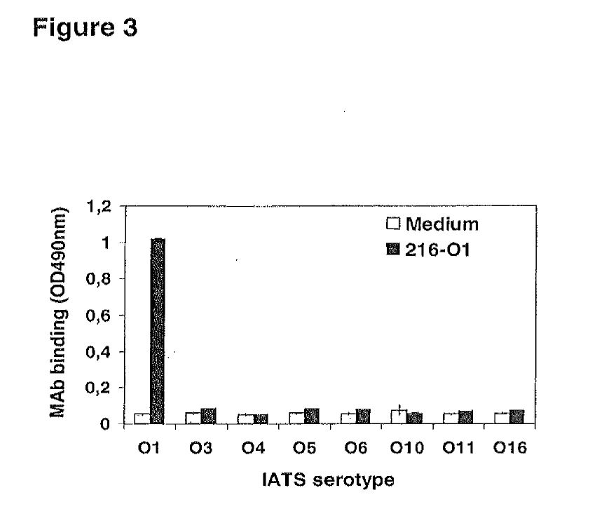

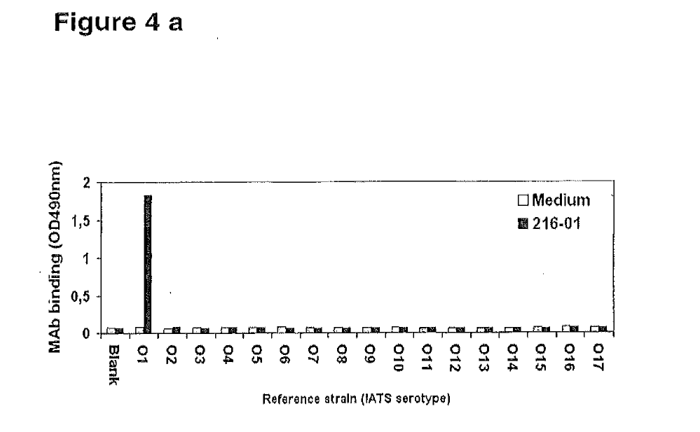

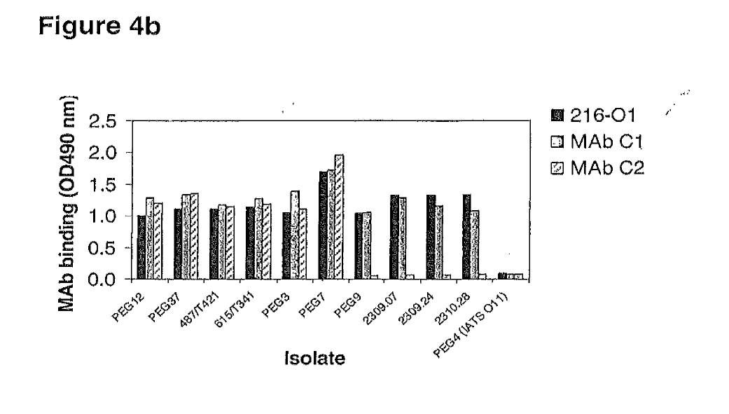

216-O1 has been generated by immunizing a healthy volunteer with an octavalent OPS-Toxin A vaccine. The vaccine contains LPS of the IATS O1 strain PA53. To determine the LPS specificity, 216-O1 was tested on a panel of isolated LPS (table 1) from P. aeruginosa (FIG. 3). To investigate whether 216-O1 specifically recognises IATS O1 P. aeruginosa, it was tested on 17 reference strains (FIG. 4a).

In addition, different clinical isolates of serotype IATS O1 (FIG. 4b) were then tested for binding to 216-O1 and other anti-P. aeruginosa LPS IATS O1 antibodies (MAb C1 and MAb C2) by whole cell ELISA. The serotype of all isolates was determined using a commercially available serotype agglutination kit and confirmed by PCR.

216-O1 reacted specifically with isolated LPS of the IATS O1 serotype, but not with any other tested serotype. Furthermore, binding was exclusively observed to the IATS O1 reference strain but not to IATS O2-O17 reference strains. Integrity of these isolates was assured using some other monoclonal antibodies against the respective serotype as positive controls (data not shown). Comparing the recognition of clinical isolates of 216-O1 with two known antibodies (MAb C1 and MAB C2), 216-O1 and MAb C1 show binding to all 10 tested clinical isolates whereas for MAb C2 only binding to 6 of the 10 tested isolates was detected.

### Example 3

### In Vitro Activity of 216-O1: Opsonophagocytic Activity

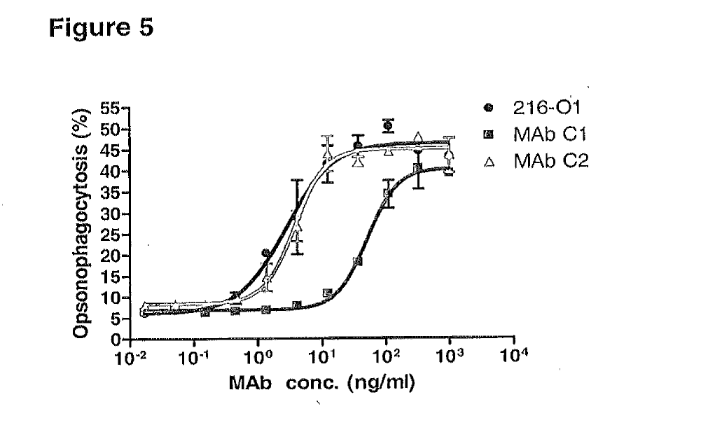

The in vitro biological activity of 216.01 was assessed using a flow cytometry-based opsonophagocytosis assay. Fluorescence-labelled ((5(6)-FAM SE)-conjugated P. aeruginosa of serotype IATS O1 were incubated with serially diluted 216-O1 in the presence of normal rabbit serum as a complement source. The opsonised bacteria were incubated with differentiated HL-60 cells (a promyelocytic cell line, ATCC: CCL-240; differentiation to phagocytes was achieved by the addition of 0.1M dimethylformamide for 4 days). Opsonophagocytosis was analysed by FACS. Positive opsonphagocytotic activity was determined by analysing the green fluorescence of the HL-60 cells in comparison with background staining of (5(6)-FAM SE)-conjugated bacteria with HL-60 cells in the absence of active complement (heat inactivated serum). The mean results of 2 independent experiments are shown in FIG. 5.

216-O1 mediated phagocytosis of P. aeruginosa of IATS O1 serotype in a dose-dependent manner (filled circles). Opsonophagocytotic activity (OA₅₀) of 216-O1, defined as the concentration resulting in the half-maximal percentage of FITC-positive HL-60 cells, was about 2.7 ng/ml. Activity at such a low dose indicates high effector potential of 216-O1. Comparing the capacity to mediate opsonophagocytosis of 216-O1 with MAb C1 (squares) and MAb C2 (triangles) a comparative opsonophagocytotic activity was detected with respect to MAb O2 (3.8 ng/ml) MAb C1 turned out to be much less effective (50.9 ng/ml).

As a result the 216-O1 antibody shows significant better characteristics in recognition of patient isolates as well as good results in opsonophagocytotic activity.

### Example 4

### In Vivo Protective Capacity of the Monoclonal Antibody 216-O1

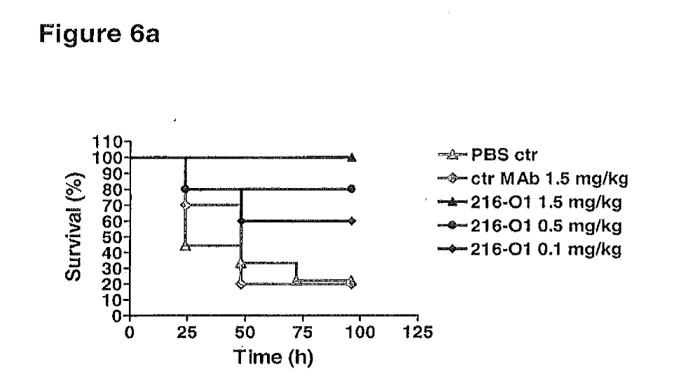

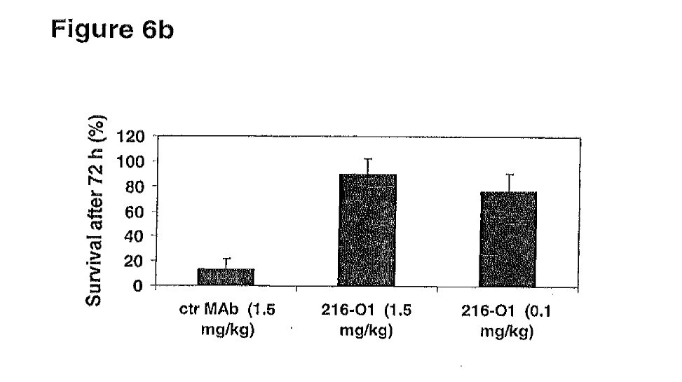

In vivo protective capacity of 216-01 was assessed in a murine burn wound sepsis model. Different doses of 216-01 were administered i.v. to NMRI mice. After two hours, a 2×2 cm burn wound was inflicted and 2.5×10⁵-5×10⁵ CFU P. aeruginosa strain PA53 (O1) were injected s.c. under the burned skin area. Mice received analgesics during the entire experimental period. Survival was monitored three times daily. One experiment showing survival rates up to 96 h after challenge (FIG. 6A) and survival rates three days after challenge of 3 independent experiments are shown (FIG. 6B).

Doses of ≧0.1 mg/kg body weight conferred 60-100% protection from systemic Pseudomonas challenge. A control antibody directed against another P. aeruginosa serotype did not confer protection. Administration of decreasing doses resulted in lower survival rates. Death was a direct result of Pseudomonas infection since mice with burn wounds but no Pseudomonas infection had a 100%-survival rate. These data demonstrate the in vivo efficacy of 216-01 against infection with P. aeruginosa.

1. A human monoclonal antibody specific for lipopolysaccharide (LPS) of the P. aeruginosa LPS serotype IATS O1 wherein the variable region of the light chain of the antibody comprises SEQ ID NO:1 in the CDR1 region, SEQ ID NO:2 in the CDR2 region and SEQ ID NO:3 in the CDR3 region, and wherein the variable region of the heavy chain of the antibody comprises SEQ ID NO:4 in the CDR1 region, SEQ ID NO:5 in the CDR2 region and SEQ ID NO:6 in the CDR3 region, or a fragment or derivative thereof capable of binding to the LPS.

2. The human monoclonal antibody of claim 1 wherein the variable region of the light chain of the antibody has the amino acid sequence of SEQ ID NO:7 and the variable region of the heavy chain has the amino acid sequence of SEQ ID NO:8; or a variant of the antibody capable of binding the LPS wherein the amino acid sequence of the variable region of the light chain of the antibody is at least 85% homologous to SEQ ID NO:7 and the amino acid sequence of the variable region of the heavy chain of the antibody is at least 85% homologous to SEQ ID NO:8.

3. The human monoclonal antibody of claim 1 wherein the light chain is of the kappa type.

4. The human monoclonal antibody of claim 1 wherein the light chain is of the lambda type.

5. The human monoclonal antibody of claim 1 wherein the heavy chain is of the IgM, IgA or IgG type.

6. The human monoclonal antibody of claim 5 wherein the heavy chain is of the IgM type.

7. The human monoclonal antibody of claim 1 wherein the antibody consists entirely of human amino acid sequence.

8. The human monoclonal antibody of claim 1 wherein the antibody exhibits human antigen recognition.

9. The human monoclonal antibody of claim 1 wherein the derivative is a mutein of the human monoclonal antibody carrying at least one conservative substitution in any of the CDR regions in the heavy or light chain.

10. The human monoclonal antibody of claim 1 wherein the antibody is N-terminally, internally or C-terminally modified.

11. The human monoclonal antibody of claim 10 wherein the modification is selected from at least one of the group consisting of oligomerization, and conjugation to a drug or a label.

12. The human monoclonal antibody of claim 1 obtainable from a human B cell or a hybridoma obtained by fusion of the human B cell with a myeloma or heteromyeloma cell.

13. A hybridoma capable of producing the human monoclonal antibody of claim 1.

14. A nucleic acid encoding the light chain of the human monoclonal antibody of claim 1 or the heavy chain of the human monoclonal antibody of claim 1.

15. (canceled)

16. A vector comprising at least one nucleic acid of claim 14.

17. The vector of claim 16, wherein the vector also comprises a promoter operatively linked to the nucleic acid to facilitate expression thereof.

18. A host cell comprising the vector of claim 16 or the nucleic acid of claim 14.

19. A method for producing the human monoclonal antibody of claim 1 comprising culturing the hybridoma of claim 13 under conditions allowing for secretion of an antibody or culturing the host cell of claim 18 under conditions suitable for expression of the human monoclonal antibody, and optionally purifying the antibody from the culture supernatant.

20. (canceled)

21. A method of performing prophylaxis or treatment of a P. aeruginosa infection in a human patient, comprising administering to the patient a human monoclonal antibody of claim 1 or a nucleic acid of claim 14.

22. The method of claim 21, wherein the human monoclonal antibody of claim 1 or the nucleic acid of claim 14 is formulated in a pharmaceutical composition.

23. The method of claim 21, wherein the P. aeruginosa infection is a hospital-acquired infection.

24. A test kit for diagnosis of a P. aeruginosa in a sample comprising at least one human monoclonal antibody of claim 1 or a nucleic acid of claim 14, and optionally further suitable ingredients for carrying out the diagnostic test.

25. The human monoclonal antibody of claim 1, wherein the antibody is formulated in a pharmaceutical composition, wherein the composition optionally comprises a pharmaceutically acceptable carrier or ingredient.

26. The nucleic acid of claim 14, wherein the nucleic acid is formulated in a pharmaceutical composition, wherein the composition optionally comprises a pharmaceutically acceptable carrier or ingredient.


SEQUENCE LISTING (16 sequences)

SEQ ID NO: 1 - protein, 16 aa, Homo sapiens
  misc_feature: CDR1 light chain
RSSQSLVYSDGNTYLN

SEQ ID NO: 2 - protein, 7 aa, Homo sapiens
  misc_feature: CDR2 light chain
KVSNRDS

SEQ ID NO: 3 - protein, 8 aa, Homo sapiens
  misc_feature: CDR3 light chain
MQGTLPFT

SEQ ID NO: 4 - protein, 5 aa, Homo sapiens
  misc_feature: CDR1 heavy chain
GFWMS

SEQ ID NO: 5 - protein, 17 aa, Homo sapiens
  misc_feature: CDR2 heavy chain
NIKEDGSLKNYVDSVKG

SEQ ID NO: 6 - protein, 6 aa, Homo sapiens
  misc_feature: CDR3 heavy chain
SAWCTY

SEQ ID NO: 7 - protein, 111 aa, Homo sapiens
  misc_feature: variable region light chain
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTLPFTFGPGTKVDIK

SEQ ID NO: 8 - protein, 115 aa, Homo sapiens
  misc_feature: variable region heavy chain
EVQLVESGGGLVQPGGSLRLSCAASGFTFSGFWMSWVRQAPGRGLEWVANIKEDGSLKNYVDSVKGRFTISRDNAENSLFLQMNSLRAEDTAVYYCCSSAWCTYWGQGTLVTVSS

SEQ ID NO: 9 - DNA, 333 bp, Homo sapiens
  misc_feature: variable region light chain
gatgttgtgatgactcagtctccactctccctgcccgtcacccttggacagccggcctccatctcctgcaggtctagtcaaagcctcgtatacagtgatggaaacacctacttgaattggtttcagcagaggccaggccaatctccgaggcgcctaatttataaggtttctaaccgggactctggggtcccagacagattcagcggcagtgggtcaggcactgatttcacactgaaaatcagcagggtggaggctgaggatgttggggtttattactgcatgcaaggtacactccctttcactttcggccctgggaccaaagtggatatcaaa

SEQ ID NO: 10 - DNA, 345 bp, Homo sapiens
  misc_feature: variable region heavy chain
gaggtgcagctggtggagtctgggggaggcttggtccagcctggggggtccctgagactctcctgtgcagcctctggattcacatttagtgggttttggatgagctgggtccgccaggctccagggagagggctggagtgggtggccaacataaaagaagatggaagtctgaaaaactatgtggactctgtgaagggccgattcaccatctccagagacaacgccgaaaactcactgtttctgcaaatgaacagcctgagagccgaggacacggctgtgtattactgctgtagttccgcctggtgcacctactggggccagggaaccctggtcaccgtctcctca

SEQ ID NO: 11 - DNA, 21 bp, Artificial Sequence
  PCR-primer
aagggttggggcggatgcact

SEQ ID NO: 12 - DNA, 23 bp, Artificial Sequence
  PCR-primer
gaagacagatggtgcagccacag

SEQ ID NO: 13 - DNA, 20 bp, Artificial Sequence
  PCR-primer
atggagtttgggctgagctg

SEQ ID NO: 14 - DNA, 20 bp, Artificial Sequence
  PCR-primer
caatgaggctccctgctcag

SEQ ID NO: 15 - DNA, 20 bp, Artificial Sequence
  Sequencing-primer
agtctgagagccgaggacac

SEQ ID NO: 16 - DNA, 19 bp, Artificial Sequence
  Sequencing-primer
acagattcagcggcagtgg